(19) United States
(12) Patent Application Publication
(10) Pub. No.: US 2018/0142365 A1
(43) Pub. Date: May 24, 2018

(54) METHOD FOR REDUCING CARBON DIOXIDE ELECTROCHEMICALLY TO GENERATE ETHYLENE SELECTIVELY

(71) Applicant: Panasonic Intellectual Property Management Co., Ltd., Osaka (JP)

(72) Inventors: SHOKO KUSAMA, Kyoto (JP); SATOSHI YOTSUHASHI, Osaka (JP); AKIHIRO SAKAI, Nara (JP); HIROSHI HASHIBA, Kyoto (JP); SHINYA OKAMOTO, Kyoto (JP); HIROKI SATO, Nara (JP); KOSUKE NAKAJIMA, Osaka (JP)

(21) Appl. No.: 15/693,475

(22) Filed: Sep. 1, 2017

(30) Foreign Application Priority Data
Nov. 24, 2016 (JP) 2016-227971
Mar. 1, 2017 (JP) 2017-038339

(51) Int. Cl. C25B 11/04; C25B 3/04; C25B 9/08; C07D 487/22

(52) U.S. Cl. CPC C25B 11/0489; C25B 3/04; C25B 9/08; C07B 2200/13; C25B 11/0405; C07D 487/22; C25B 11/0415

(57) ABSTRACT

The present invention provides a method for reducing carbon dioxide electrochemically to generate ethylene selectively. In the present method, a carbon dioxide reduction catalyst comprising a crystalline copper phthalocyanine is used to generate ethylene selectively by reducing carbon dioxide electrochemically on a cathode electrode.

### BACKGROUND

### 1. Technical Field

The present disclosure relates to a method for reducing carbon dioxide electrochemically to generate ethylene selectively. The present disclosure also relates to an electrolysis device, a carbon dioxide reduction electrode, and a carbon dioxide reduction catalyst used for the method.

### 2. Description of the Related Art

Recently, metal phthalocyanine has been known as a catalyst capable of reducing carbon dioxide electrochemically. The performance of the metal phthalocyanine has also been analyzed.

Furuya discloses a method for reducing carbon dioxide electrochemically using a gas diffusion electrode including cobalt phthalocyanine as a cathode electrode in Japanese Patent Application Laid-open Publication No. Hei 1-205088.

Molter, Trent M. discloses a method for reducing carbon dioxide using a cathode electrode including copper phthalocyanine in a solid polymer electrolyte in European Patent Specification No. EP 0 390 157 B1 and U.S. Pat. No. 4,921,585.

Furuya et al. discloses a method for reducing carbon dioxide electrochemically using a gas diffusion electrode on which a mixture of copper phthalocyanine and carbon black has been applied as a cathode electrode in their article “Electroreduction of carbon dioxide on gas-diffusion electrodes modified by metal phthalocyanines”, Journal of electroanalytical chemistry and interfacial electrochemistry 271.1 (1989): 181-191.

### SUMMARY

The present invention provides a method for reducing carbon dioxide electrochemically to generate ethylene selectively, the method comprising:

(a) preparing an electrolysis device comprising:

a cathode container;

an anode container;

a cathode electrode;

an anode electrode; and

a solid electrolysis membrane;

wherein

a first electrolysis solution is stored in the cathode container;

the first electrolysis solution contains the carbon dioxide;

a second electrolysis solution is stored in the anode container;

the cathode electrode is in contact with the first electrolysis solution;

the cathode electrode comprises a carbon dioxide reduction catalyst;

the carbon dioxide reduction catalyst comprises a crystalline copper phthalocyanine;

the anode electrode is in contact with the second electrolysis solution; and

the cathode container and the anode container are separated from each other with the solid electrolysis membrane; and

(b) applying a voltage to the cathode electrode and the anode electrode in such a manner that an electric potential of the cathode electrode is negative with regard to an electric potential of the anode electrode to generate ethylene selectively due to electrochemical reduction of the carbon dioxide on the cathode electrode.

The present disclosure provides a method for reducing carbon dioxide electrochemically to generate ethylene selectively.

### BRIEF DESCRIPTION OF THE DRAWINGS

FIG. 1 shows a schematic view of an electrolysis device according to the embodiment of the present disclosure.

FIG. 2 shows a schematic view of a cathode electrode according to the embodiment of the present disclosure.

FIG. 3 shows a powder X-ray diffraction profile of commercially available copper phthalocyanine β-type crystalline powder used in the inventive example 1.

FIG. 4 shows a powder X-ray diffraction profile of copper phthalocyanine-carbon black hybrid catalyst used in the inventive example 1.

FIG. 5 shows a powder X-ray diffraction profile of commercially available copper phthalocyanine α-type crystalline powder used in the inventive example 2.

FIG. 6 shows a powder X-ray diffraction profile of copper phthalocyanine-carbon black hybrid catalyst used in the inventive example 2.

FIG. 7 shows a powder X-ray diffraction profile of copper phthalocyanine-carbon black hybrid catalyst used in the inventive example 3.

FIG. 8 shows a powder X-ray diffraction profile of commercially available 1,2,3,4,8,9,10,11,15,16,17,18,22,23,24,25-hexadecafluorophthalocyanine copper crystalline powder used in the inventive example 4.

FIG. 9 shows a powder X-ray diffraction profile of copper phthalocyanine-carbon black hybrid catalyst used in the inventive example 4.

FIG. 10 shows a powder X-ray diffraction profile of commercially available 2,3,9,10,16,17,23,24-octafluorophthalocyanine copper crystalline powder in the inventive example 5.

FIG. 11 shows a powder X-ray diffraction profile of copper phthalocyanine-carbon black hybrid catalyst used in the inventive example 5.

FIG. 12 shows a powder X-ray diffraction profile of commercially available 2,9,16,23-tetra-tert-butyl phthalocyanine copper crystalline powder in the inventive example 6.

FIG. 13 shows a powder X-ray diffraction profile of copper phthalocyanine-carbon black hybrid catalyst used in the inventive example 6.

FIG. 14 shows a powder X-ray diffraction profile of commercially available 5,9,14,18,23,27,32,36-octabutoxy-2,3-naphthalocyanine copper crystalline powder in the inventive example 7.

FIG. 15 shows a powder X-ray diffraction profile of copper phthalocyanine-carbon black hybrid catalyst used in the inventive example 7.

FIG. 16 shows a powder X-ray diffraction profile of copper phthalocyanine-carbon black hybrid catalyst used in the comparative example 1.

FIG. 17 shows a powder X-ray diffraction profile of copper phthalocyanine-carbon black hybrid catalyst used in the comparative example 2.

### DETAILED DESCRIPTION OF THE EMBODIMENT

Furuya fails to disclose an experiment result in a case where copper phthalocyanine is used as a catalyst in Japanese Patent Application Laid-open Publication No. Hei 1-205088. Neither Molter, Trent M. nor Furuya et al. discloses presence or absence of crystallinity of copper phthalocyanine. The presence or absence of the crystallinity remains unknown.

Copper phthalocyanine is known to be classified in plural crystal forms on the basis of its diffraction angle in the X-ray diffraction spectrum. Characteristic crystal forms of copper phthalocyanine include at least three kinds of α-type crystal form, β-type crystal form, and a γ-type crystal form. Among them, intensive research has been conducted on the crystalline structures of the stable β-type crystal form and the metastable α-type crystal form. However, no report has not issued on the relation between the crystallinity of the copper phthalocyanine and its performance of carbon dioxide reduction.

In the method for reducing carbon dioxide disclosed in Molter, Trent M. and Furuya et al., main products are carbon monoxide and formic acid provided through two-electron reduction reaction. Therefore, there is a problem that ethylene, which is useful, fails to be generated through multi-electron reduction reaction.

Hereinafter, an embodiment of the present disclosure will be described with reference to the drawings.

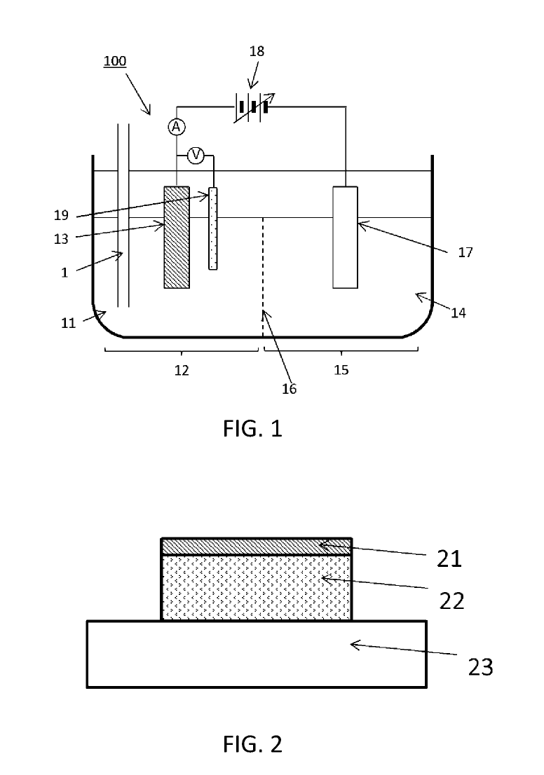

FIG. 1 shows a schematic view of an electrolysis device 100 according to the embodiment of the present disclosure. The electrolysis device 100 comprises a cathode container 12 for storing a first electrolyte solution 11 containing an electrolysis reactant, a cathode electrode 13 having crystalline copper phthalocyanine disposed in the cathode container 12 so as to be in contact with the first electrolyte solution 11, an anode container 15 for storing a second electrolyte solution 14, a solid electrolyte membrane 16 for separating the cathode container 12 and the anode container 15 from each other, an anode electrode 17 having a region formed of a metal or a metal compound disposed in the anode container 15 so as to be in contact with the second electrolyte solution 14, an external power source 18 for applying a voltage between the cathode electrode 13 and the anode electrode 17 in such a manner that the electric potential of the cathode electrode 13 is negative with regard to the electric potential of the anode electrode 17, and a reference electrode 19 disposed in the cathode container 12 so as to be in contact with the first electrolyte solution 11.

In the present embodiment, carbon dioxide is reduced electrochemically in a state where the cathode electrode 13 contains copper phthalocyanine in which the crystallinity thereof is maintained. Therefore, ethylene is generated selectively. Furthermore, since the electrolytic reaction is controlled by controlling the electric potential of the cathode electrode 13, the anode electrode 17 is prevented from being deteriorated with time. For this reason, the present embodiment provides a desirable electrolysis device.

As shown in FIG. 1, the cathode container 12 may be provided with a pipe 1 in the electrolysis device 100. A gaseous electrolysis reactant is supplied to the first electrolyte solution 11 through the pipe 1. A gas other than carbon dioxide may be reduced using the electrolysis device. An example of such a gas is oxygen and nitrogen. Furthermore, the electrolysis device 100 may be used for a liquid or solid electrolysis reactant such as water. In this case, an inert gas such as nitrogen or argon is supplied through a pipe provided separately from the pipe 1 to prevent a side reaction. One end of the pipe 1 is immersed in the first electrolyte solution 11. The electrolysis device 100 may comprise a voltage measurement device and an electric-current measurement device to monitor how to reduce the electrolysis reactant. Carbon dioxide is reduced electrochemically using the electrolysis device 100 to generate ethylene selectively. An example of the electrolysis products other than ethylene is hydrogen, carbon monoxide, methane, or formic acid.

The cathode electrode 13 has a mixture of crystalline copper phthalocyanine and carbon black, namely, a crystalline copper phthalocyanine-carbon black hybrid catalyst. Hereinafter, the word “cathode electrode” is referred to as “carbon dioxide reduction electrode”. Crystalline copper phthalocyanine may be purchased commercially or be synthesized. In the synthesis method, for example, a vacuum deposition method, an ion beam deposition method, or a solvent-milling method may be employed. Alternatively, in the synthesis method, copper phthalocyanine is evaporated at a low pressure in an inert gas. Crystalline copper phthalocyanine is not limited to copper phthalocyanine which does not have a substituent. Crystalline copper phthalocyanine may be a compound in which at least one substituent have been introduced. An example of such a compound is 1,2,3,4,8,9,10,11,15,16,17,18,22,23,24,25-hexadeca fluorophthalocyanine copper, 2,3,9,10,16,17,23,24-octafluorophthalocyanine copper, 2,9,16,23-tetra-tert-butylphthalocyanine copper, or 5,9,14,18,23,27,32,36-octabutoxy-2,3-naphthalocyanine copper. Crystalline copper phthalocyanine is not limited to the above examples. As long as an electrolysis product is provided due to catalyst action through the crystalline copper phthalocyanine, the chemical structure of the crystalline copper phthalocyanine is not limited.

The concentration of the copper phthalocyanine to be mixed with carbon black is set freely. As the concentration is higher, a surface area of copper phthalocyanine which adsorbs on the surface of carbon black is also larger. Therefore, the catalyst activity is improved. However, when the concentration is too high, the intensity of carbon black is lowered. This would cause the decrease in the catalyst activity. To solve this problem, desirably, the concentration of copper phthalocyanine in carbon black is, for example, approximately 44%. However, as long as the electrolysis product is provided due to the catalyst activity through crystalline copper phthalocyanine, the concentration is not limited.

Hereinafter, one example of synthesis methods of a crystalline copper phthalocyanine-carbon black hybrid catalyst included in the cathode electrode 13 will be described.

Copper phthalocyanine and carbon black may be dispersed in a solvent. An example of the solvent is N,N-dimethylformamide, acetone, ethanol, 1-propanol, or ethyl acetate. The solvent may be one selected from these materials and may contain two or more kinds of these materials. The solvent is not limited to the above-exemplified materials.

The cathode electrode 13 may be composed only of the crystalline copper phthalocyanine-carbon black hybrid catalyst. Alternatively, the cathode electrode 13 may have a stacked structure of a substrate for supporting the crystalline copper phthalocyanine-carbon black hybrid catalyst and an electric conductive layer for improving electric conductivity of the electrode. For example, as shown in FIG. 2, the cathode electrode 13 has a structure comprising the crystalline copper phthalocyanine-carbon black hybrid catalyst 21, the electric conductive layer 22 on which the crystalline copper phthalocyanine-carbon black hybrid catalyst has been applied, and the substrate 23 onto which the electric conductive layer 22 has been adhered with an electric conductive paste. In such a structure, since the electrolytic solution passes through the crystalline copper phthalocyanine-carbon black hybrid catalyst 21 due to its structural property, the electric conductive layer 22 and the substrate 23 must not be brought into contact with the electrolyte solution. Alternatively, it is required to use the electric conductive layer 22 and the substrate 23 which are inactive as catalysts. An example of the material of the electric conductive layer 22 is carbon or metal. An example of the substrate 23 is a glass substrate, an epoxy resin substrate, or a carbon substrate such as a substrate in which a glassy carbon has been employed. In light of both of the electric conductivity and the catalyst inactivity, it is desirable that the substrate 23 is a carbon substrate. To improve electric property of the cathode electrode 13, it is desirable that the crystalline copper phthalocyanine-carbon black hybrid catalyst 21 is immobilized on the electric conductive layer 22. In the desirable immobilization method, for example, the crystalline copper phthalocyanine-carbon black hybrid catalyst 21 is pressed on the electric conductive layer 22 and a binder of a solution in which a Nafion is dispersed is used. As long as the cathode electrode 13 has an activity of reducing carbon dioxide, the constitution of the cathode electrode 13 is not limited.

The cathode electrode 13 is in contact with the first electrolyte solution 11. More exactly, the crystalline copper phthalocyanine-carbon black hybrid catalyst 21 comprised in the cathode electrode 13 is in contact with the first electrolyte solution 11. Only a part of the cathode electrode 13 may be immersed in the first electrolyte solution 11, as far as the crystalline copper phthalocyanine-carbon black hybrid catalyst 21 is in contact with the first electrolyte solution 11.

The anode electrode 17 comprises an electrically conductive material. An example of the electrically conductive material is carbon, platinum, gold, silver, copper, titanium, iridium oxide or the alloy thereof. Unless the electrically conductive material is decomposed due to the oxidation reaction of itself, the material of the electrically conductive material is not limited.

The oxidation reaction of water at the anode electrode 17 is a reaction system independent from the reduction reaction of carbon dioxide at the cathode electrode 13. For this reason, the material of the anode electrode 17 does not have an effect on the reaction which occurs at the cathode electrode 13.

The anode electrode 17 is in contact with the second electrolyte solution 14. More exactly, the electrically conductive material comprised in the anode electrode 17 is in contact with the second electrolyte solution 14. Only a part of the anode electrode 17 may be immersed in the second electrolyte solution 14, as far as the electrically conductive material is in contact with the second electrolyte solution 14.

The first electrolyte solution 11 is stored in the cathode container 12. The first electrolyte solution 11 is an electrolyte solution having a predetermined concentration. An example of the electrolyte solution is a potassium chloride aqueous solution or a potassium hydrogen carbonate aqueous solution. The second electrolyte solution 14 is stored in the anode container 15. The second electrolyte solution 14 is an electrolyte solution having a predetermined concentration. An example of the electrolyte solution is a potassium hydrogen carbonate aqueous solution or a sodium hydroxide aqueous solution. The upper limit of the concentration of the electrolyte solution is determined depending on saturation concentration of the electrolyte. Generally, the electrolyte solution has a concentration of not less than 0.1 mol/l and not more than 0.3 mol/l.

The solid electrolyte membrane 16 is required to separate the cathode container 12 for storing the first electrolyte solution 11 and the anode container 15 for storing the second electrolyte solution 14 from each other and to prevent the components of these electrolyte solutions from being mixed with each other. Since protons pass through the solid electrolyte membrane 16, the first electrolyte solution 11 in contact with the cathode electrode 13 is electrically connected with the second electrolyte solution 14 in contact with the anode electrode 17. For example, the solid electrolyte membrane 16 is a Nafion film which is commercially available from DuPont.

The reference electrode 19 is used to measure the electric potential of the cathode electrode 13 and is connected to the cathode electrode 13 through a voltage measurement device. An example of the reference electrode 19 is a silver/silver chloride electrode.

The above-mentioned embodiment is a two-liquid system in which the cathode container 12 for storing the first electrolyte solution 11 and the anode container 15 for storing the second electrolyte solution 14 are separated from each other with the solid electrolyte membrane 16. In this two-liquid system, for example, in a case where both of the first electrolyte solution 11 and the second electrolyte solution 14 are sodium chloride aqueous solutions, an electrode on which a harmful chlorine gas is not generated on the anode electrode 17 at the electrolysis reaction on the cathode electrode 13 is required to be selected as the anode electrode 17. In a one-liquid system in which the solid electrolyte membrane 16 is absent, reverse reaction may occur in which the electrolysis product which has been generated in the cathode container 12 is oxidized back to the electrolysis reactant. Therefore, another contraption for removing the electrolyte product immediately from the reaction system is required such as a liquid circulation system constituted outside.

(Method for Generating the Electrolyte Product)

Hereinafter, a method for generating the electrolyte product using the above-mentioned electrolysis device 100 will be described.

A user prepares the electrolysis device 100. Concretely speaking, the user may purchase the electrolysis device 100. Alternatively, the user may assemble the electrolysis device 100. The electrolysis device 100 may be disposed at room temperature under atmospheric pressure; however, a cell operable under high pressure may be used to go ahead with carbon dioxide reduction reaction more rapidly.

The external power source 18 applies a voltage between the cathode electrode 13 and the anode electrode 17 in such a manner that the electric potential of the cathode electrode 13 is negative with regard to the electric potential of the anode electrode 17. The voltage applied by the external power source 18 is equal to or more than the threshold necessary for providing the generation reaction of the electrolyte product. The threshold is changed depending on the distance between the cathode electrode 13 and the anode electrode 17, the types of the materials of the cathode electrode 13 and the anode electrode 17, or the concentration of the first electrolyte solution 11.

A part of the voltage applied between the cathode electrode 13 and the anode electrode 17 is spent for oxidation reaction of water on the anode electrode 17. Using the electrolysis device 100 shown in FIG. 1, the voltage which is being applied actually to the cathode electrode 13 is measured more exactly. The electric potential of the cathode electrode 13 with regard to the electric potential of the reference electrode 19 is changed depending on the type of the material of the reference electrode 19. For example, when the reference electrode 19 is a silver/silver chloride electrode, the electric potential of the cathode electrode 13 with regard to the electric potential of the reference electrode 19 is, usually, not more than −0.2 volts in the carbon dioxide reduction reaction, not more than −0.0 volts in a hydrogen generation reaction, and not more than 1.2 volts in an oxygen generation reaction.

As just described, a suitable voltage is applied to the cathode electrode 13 to reduce the electrolysis reactant contained in the first electrolyte solution 11 on the cathode electrode 13. As a result, the electrolysis product is generated on the surface of the cathode electrode 13.

It is desirable that the solid electrolyte membrane 16 separates the cathode container 12 and the anode container 15 from each other to prevent the first electrolyte solution 11 from being mixed with the second electrolyte solution 14.

A reaction electric current flows through the cathode electrode 13 due to the reduction reaction of the electrolysis reactant on the surface of the cathode electrode 13 using the electrolysis device 100 and due to the oxidation reaction of water on the surface of the anode electrode 17. As shown in FIG. 1, the amount of the reaction electric current can be monitored, if the electric current measurement device is installed in the electrolysis device 100.

### EXAMPLES

Hereinafter, the present disclosure will be described in more detail with reference to the following examples.

### Inventive Example 1

(Fabrication of Cathode Electrode 13)

A cathode electrode 13 containing a crystalline copper phthalocyanine-carbon black hybrid catalyst 21 was fabricated.

First, a disk-shaped glassy carbon substrate having a diameter of 10 millimeters and a thickness of 8 millimeters was adhered on a metal sheet disposed on a surface of a glass substrate. The metal sheet was formed of aluminum. Subsequently, a surface part other than a circular plate part of the glassy carbon substrate and an exposed surface of the metal sheet were covered with an epoxy resin in such a manner that these surfaces are prevented from being in contact with an electrolyte solution.

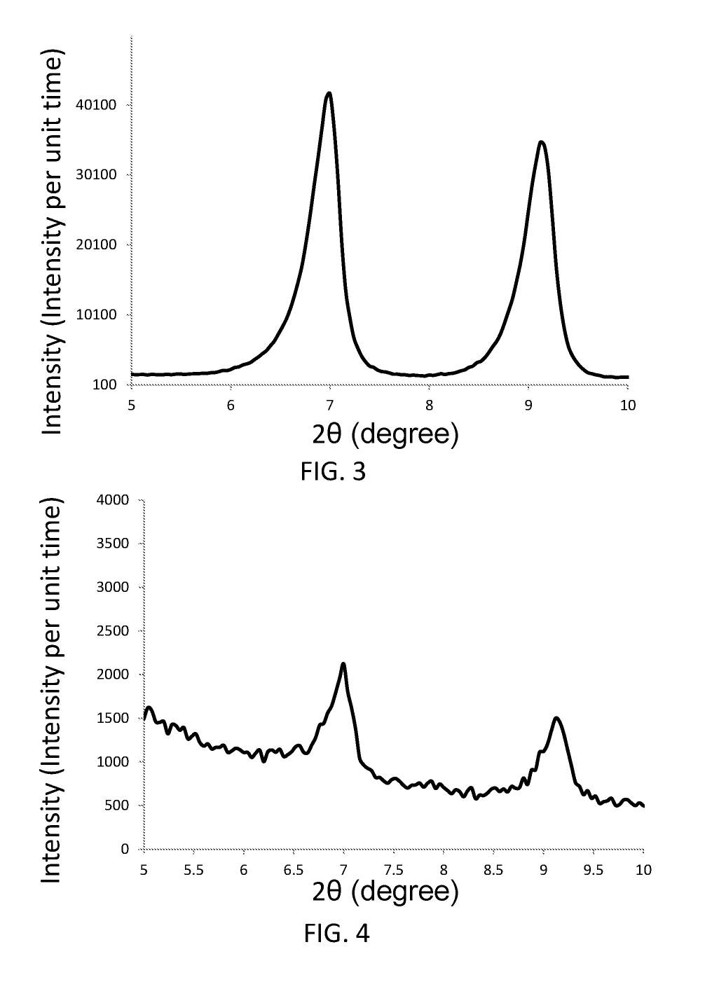

Carbon black having a mean particle size of 50 nanometers was purchased from Cabot Corporation as a trade name of Vulcan XC-72R. Copper phthalocyanine β-type crystalline powders purchased from Tokyo Chemical Industry Co., Ltd. were used as copper phthalocyanine particles. The copper phthalocyanine β-type crystalline powders exhibited diffraction peaks at 7.0° (lattice constant: 1.26 nanometers) and 9.2° (lattice constant: 0.96 nanometers) within a Bragg angle 2θ range of not less than 5° and not more than 10° in a powder X-ray diffraction method using a CuKα ray. See FIG. 3. The carbon black (150 milligrams) and the copper phthalocyanine β-type crystalline powders (66 milligrams) were dispersed in a first solvent consisting of N,N-dimethylformamide. Then, an ultrasonic wave was applied to the dispersion liquid. The N,N-dimethylformamide was removed using a rotary evaporator. In this way, a crystalline copper phthalocyanine-carbon black hybrid catalyst 21 was provided. The copper phthalocyanine content contained in the crystalline copper phthalocyanine-carbon black hybrid catalyst 21 was 44% by weight ratio.

The crystalline copper phthalocyanine-carbon black hybrid catalyst 21 was dispersed in a second solvent consisting of acetone containing a Nafion dispersion solution (purchased from Sigma-Aldrich Co., LLC.). Then, an ultrasonic wave was applied to the dispersion liquid to provide an ink solution. The ink solution was applied to the glassy carbon substrate and then dried. In this way, a cathode electrode 13 according to the present disclosure was fabricated. The copper phthalocyanine concentration on the electrode was 0.3 micromol/cm².

The crystallinity of the crystalline copper phthalocyanine-carbon black hybrid catalyst 21 applied on the glassy carbon substrate was evaluated in the power X-ray diffraction method using the CuKα ray. As a result, as shown in FIG. 4, the diffraction peaks appeared at 7.0° (lattice constant: 1.26 nanometers, half maximum full-width: 0.29°) and 9.2° (lattice constant: 0.96 nanometers, half maximum full-width: 0.31°) within a Bragg angle 2θ range of not less than 5° and not more than 10°. Therefore, the present inventors confirmed that the copper phthalocyanine contained in the catalyst was a β-type crystal.

(Assembling of Device)

The electrolysis device 100 shown in FIG. 1 was assembled using the above-fabricated cathode electrode 13. The components of the electrolysis device 100 according to the present example are listed below.

Cathode electrode 13: Crystalline copper phthalocyanine-carbon black hybrid catalyst 21/Glassy carbon substrate (Surface area: 0.785 cm²)

Anode electrode 17: Platinum

Distance between Cathode electrode 13 and Anode electrode 17: 5 centimeters

Reference electrode 19: Silver/Silver chloride

First electrolyte solution 11: Potassium chloride aqueous solution (0.5 mol/L)

Second electrolyte solution 14: Potassium hydrogen carbonate aqueous solution (3.0 mol/L)

Solid electrolyte membrane 16: Nafion membrane (product of DuPont, trade name: Nafion 424)

The first electrolyte solution 11 was bubbled for sixty minutes with a carbon dioxide gas supplied through a pipe 1 at a carbon dioxide supply rate of 125 cm³/minute. The carbon dioxide gas was dissolved in the first electrolyte solution 11.

Then, the cathode container 12 was sealed. A voltage was applied between the anode electrode 17 and the cathode electrode 13 using a potentiostat in such a manner that the electric potential of the cathode electrode 13 was negative with regard to the electric potential of the anode electrode 17. The value of the applied voltage was controlled with the potentiostat in such a manner that the electric potential of the cathode electrode 13 with regard to the reference electrode 19 was −1.6 volts.

After the voltage was applied for 10,000 seconds, the type and the amount of reaction products generated in the cathode container 12 were measured with gas chromatography and liquid chromatography. As a result, hydrogen (H₂), carbon monoxide (CO), methane (CH₄), ethylene (C₂H₄), and formic acid (HCOOH) were detected as reduction products of carbon dioxide. See Table 1. In other words, a hydrocarbon such as ethylene or methane was produced by reducing carbon dioxide on the cathode electrode 13 using the crystalline copper phthalocyanine-carbon black hybrid catalyst 21.

As a result of the experiment, the generation ratio of ethylene was 41%. See Table 1.

The generation ratio of ethylene is generation efficiency of ethylene of the generation efficiency of the whole of the provided products. The generation ratio of ethylene is calculated on the basis of (the generation ratio of ethylene)=(generation efficiency of ethylene)/(generation efficiency of the whole of the provided products)×100 [%]. Here, the whole of the provided products means hydrogen, carbon monoxide, methane, ethylene, and formic acid. The generation efficiency of ethylene means a ratio of electric charge amount used for generation of ethylene to the whole of the reaction electric charge amount. The generation efficiency of ethylene is calculated on the basis of (the generation efficiency of ethylene)=(the reaction electric charge amount used for the generation of ethylene)/(the whole of the reaction electric charge amount)×100 [%]. The generation efficiency of the whole of the products means a ratio of electric charge amount used for the generation of the whole of the products to the whole of the reaction electric charge amount. The generation efficiency of the whole of the products is calculated on the basis of (the generation efficiency of the whole of the products)=(the reaction electric charge amount used for the generation of the whole of the products)/(the whole of the reaction electric charge mount)×100 [%].

### Inventive Example 2

An experiment similar to the inventive example 1 was conducted, except that:

(I) copper phthalocyanine α-type crystalline powders (purchased from Tokyo Chemical Industry Co., Ltd.) were used as the copper phthalocyanine particles;
(II) the crystalline copper phthalocyanine-carbon black hybrid catalyst 21 was provided using 1-propanol as the first solvent;
(III) the ink solution was provided using ethanol as the second solvent; and
(IV) the electrolysis period was 10,046 seconds.

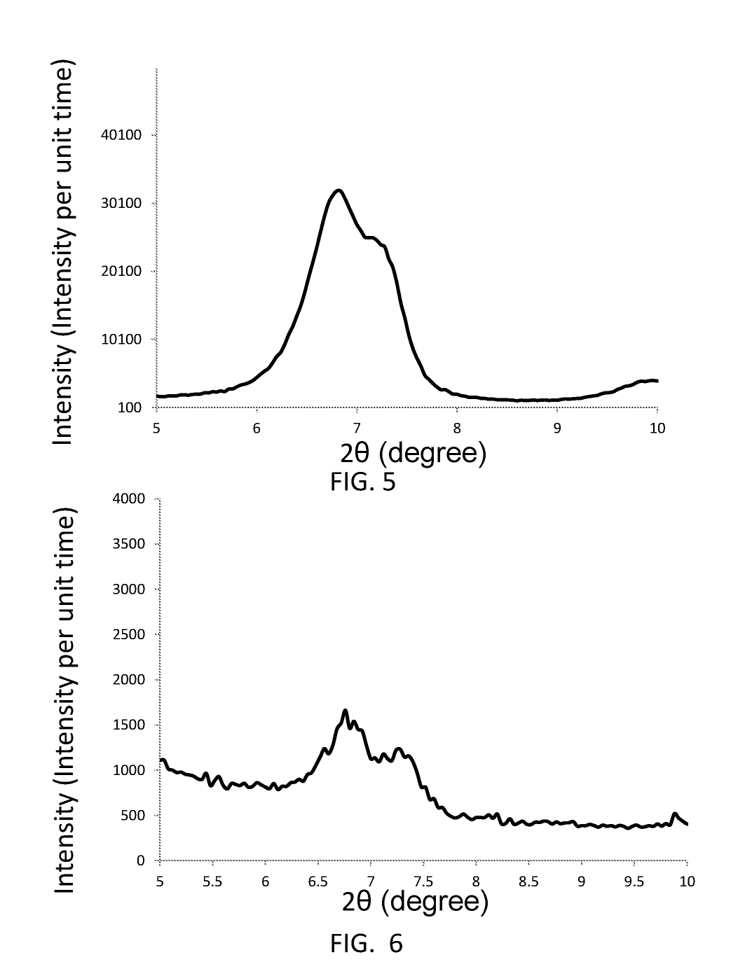

The copper phthalocyanine α-type crystalline powders exhibited diffraction peaks at 6.8° (lattice constant: 1.30 nanometers) and 7.2° (lattice constant: 1.20 nanometers) within a Bragg angle 2θ range of not less than 5° and not more than 10° in a powder X-ray diffraction method using a CuKα ray. See FIG. 5.

The crystallinity of the catalyst applied on the glassy carbon substrate was evaluated in the power X-ray diffraction method using the CuKα ray. As a result, as shown in FIG. 6, the diffraction peaks appeared at 6.8° (lattice constant: 1.30 nanometers, half maximum full-width: 0.55°) and 7.3° (lattice constant: 1.21 nanometers, half maximum full-width: 0.39°) within a Bragg angle 2θ range of not less than 5° and not more than 10°. Therefore, the present inventors confirmed that the copper phthalocyanine contained in the catalyst was an α-type crystal.

As a result of the experiment, the generation ratio of ethylene was 31%. See Table 1.

### Inventive Example 3

An experiment similar to the inventive example 1 was conducted, except that the voltage was applied in such a manner that the electric potential of the cathode electrode 13 with regard to the reference electrode 19 was −1.7 volts.

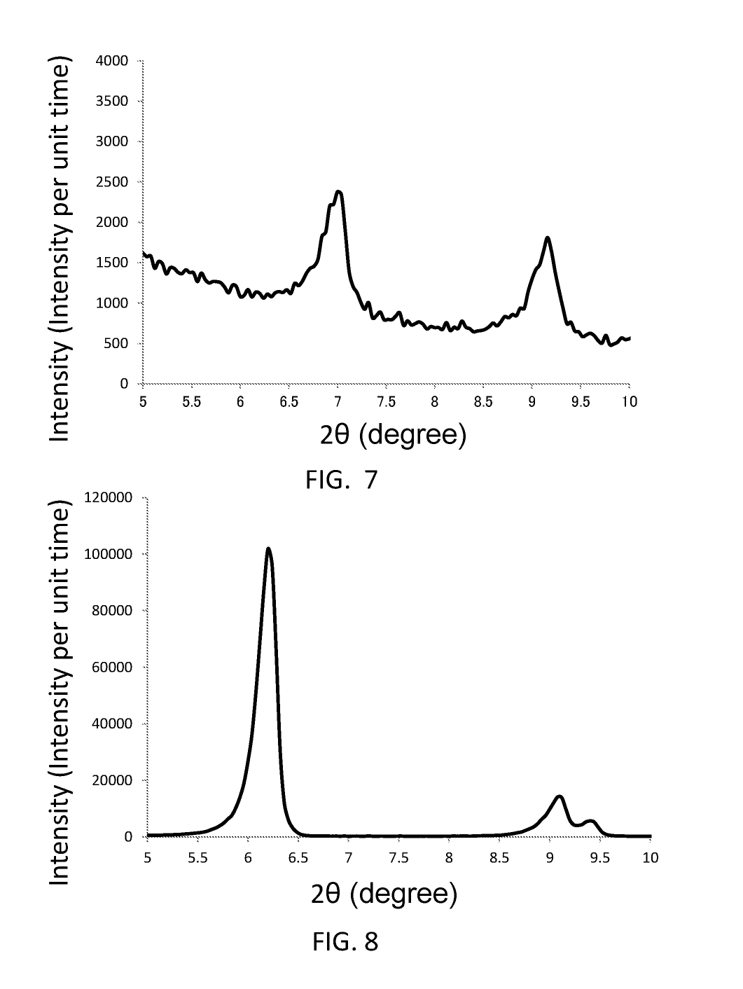

The crystallinity of the catalyst applied on the glassy carbon substrate was evaluated in the power X-ray diffraction method using the CuKα ray. As a result, as shown in FIG. 7, the diffraction peaks appeared at 7.0° (lattice constant: 1.26 nanometers, half maximum full-width: 0.29°) and 9.2° (lattice constant: 0.96 nanometers, half maximum full-width: 0.30°) within a Bragg angle 2θ range of not less than 5° and not more than 10°. Therefore, the present inventors confirmed that the copper phthalocyanine contained in the catalyst was a β-type crystal.

As a result of the experiment, the generation ratio of ethylene was 42%. See Table 1.

### Inventive Example 4

An experiment similar to the inventive example 1 was conducted, except that:

(I) 1,2,3,4,8,9,10,11,15,16,17,18,22,23,24,25-hexadecafluorophthalocyanine copper particles (purchased from Tokyo Chemical Industry Co., Ltd.) were used as the copper phthalocyanine particles;
(II) the carbon black (148 milligrams) and the 1,2,3,4,8,9,10,11,15,16,17,18,22,23,24,25-hexadecafluorophthalocyanine copper particles (103 milligrams) were mixed in the first solvent;
(III) the crystalline copper phthalocyanine-carbon black hybrid catalyst 21 was provided using ethanol as the first solvent; and
(IV) the voltage was applied in such a manner that the electric potential of the cathode electrode 13 with regard to the reference electrode 19 was −1.7 volts.

The 1,2,3,4,8,9,10,11,15,16,17,18,22,23,24,25-hexadecafluorophthalocyanine copper particles exhibited a diffraction peak at 6.2° (lattice constant: 1.41 nanometers) within a Bragg angle 2θ range of not less than 5° and not more than 10° in a powder X-ray diffraction method using a CuKα ray. See FIG. 8.

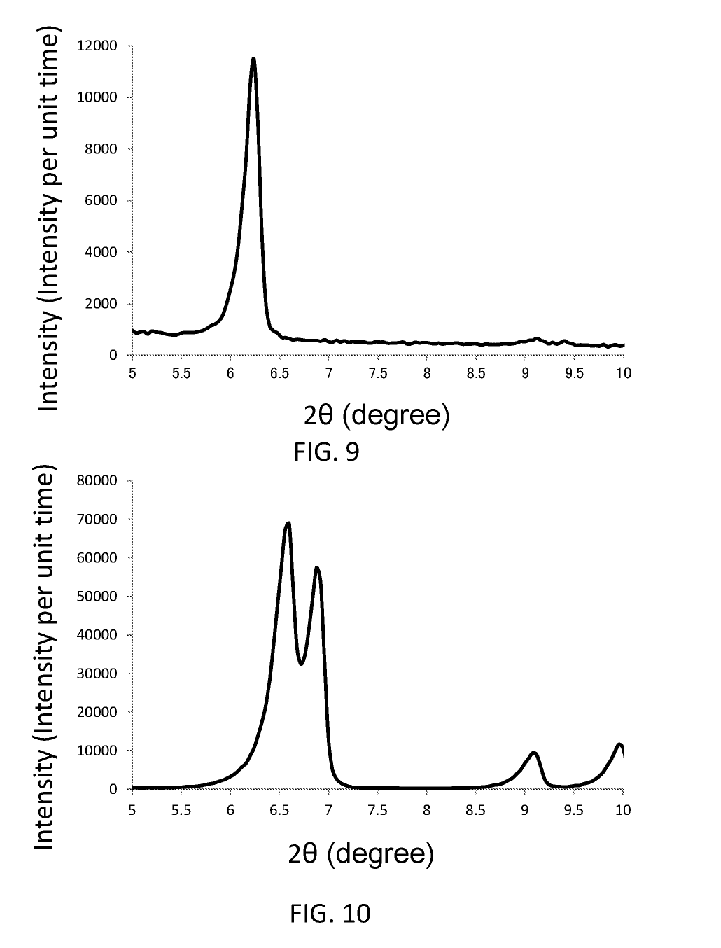

The crystallinity of the catalyst applied on the glassy carbon substrate was evaluated in the power X-ray diffraction method using the CuKα ray. As a result, as shown in FIG. 9, the diffraction peak appeared at 6.2° (lattice constant: 1.41 nanometers, half maximum full-width: 0.19°) within a Bragg angle 2θ range of not less than 5° and not more than 10°. Therefore, the present inventors confirmed that the copper phthalocyanine contained in the catalyst was a 1,2,3,4,8,9,10,11,15,16,17,18,22,23,24,25-hexadecafluorophthalocyanine copper crystal.

As a result of the experiment, the generation ratio of ethylene was 43%. See Table 1.

### Inventive Example 5

An experiment similar to the inventive example 1 was conducted, except that:

(I) 2,3,9,10,16,17,23,24-octafluorophthalocyanine copper particles (purchased from Tokyo Chemical Industry Co., Ltd.) were used as the copper phthalocyanine particles;
(II) the carbon black (150 milligrams) and the 2,3,9,10,16,17,23,24-octafluorophthalocyanine copper particles (86 milligrams) were mixed in the first solvent;
(III) the crystalline copper phthalocyanine-carbon black hybrid catalyst 21 was provided using ethanol as the first solvent; and
(IV) the voltage was applied in such a manner that the electric potential of the cathode electrode 13 with regard to the reference electrode 19 was −1.7 volts.

The 2,3,9,10,16,17,23,24-octafluorophthalocyanine copper particles exhibited diffraction peaks at 6.6° (lattice constant: 1.34 nanometers) and 6.9° (lattice constant: 1.28 nanometers) within a Bragg angle 2θ range of not less than 5° and not more than 10° in a powder X-ray diffraction method using a CuKα ray. See FIG. 10.

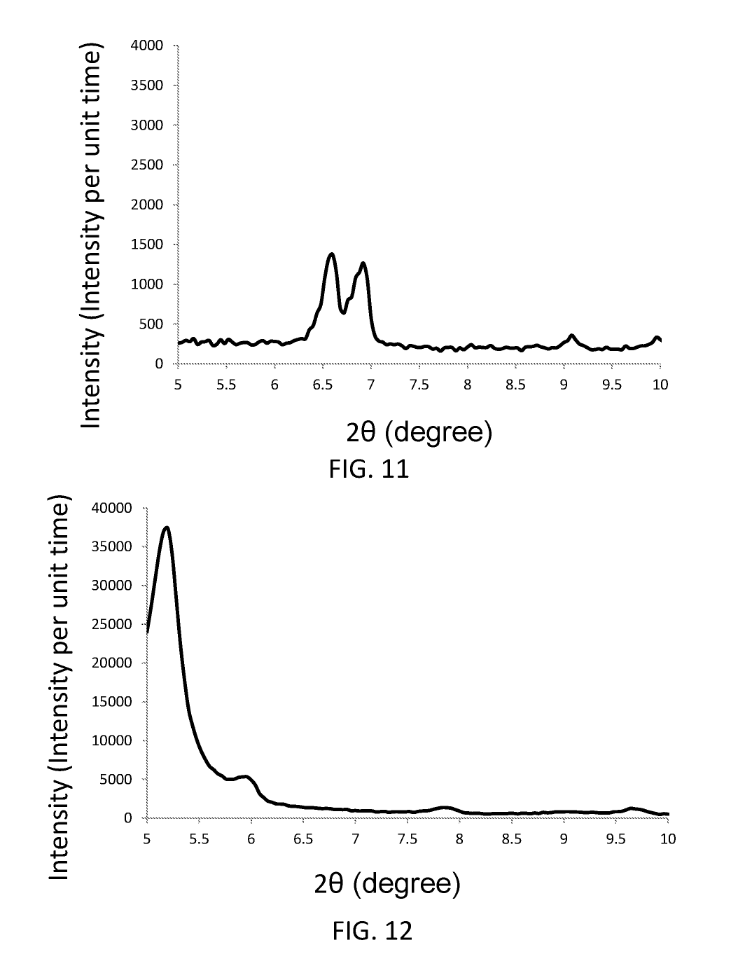

The crystallinity of the catalyst applied on the glassy carbon substrate was evaluated in the power X-ray diffraction method using the CuKα ray. As a result, as shown in FIG. 11, the diffraction peaks appeared at 6.6° (lattice constant: 1.34 nanometers, half maximum full-width: 0.17°) and 6.9° (lattice constant: 1.28 nanometers, half maximum full-width: 0.22°) within a Bragg angle 2θ range of not less than 5° and not more than 10°. Therefore, the present inventors confirmed that the copper phthalocyanine contained in the catalyst was a 2,3,9,10,16,17,23,24-octafluorophthalocyanine copper crystal.

As a result of the experiment, the generation ratio of ethylene was 38%. See Table 1.

### Inventive Example 6

An experiment similar to the inventive example 1 was conducted, except that:

(I) 2,9,16,23-tetra-tert-butylphthalocyanine copper particles (purchased from Tokyo Chemical Industry Co., Ltd.) were used as the copper phthalocyanine particles;
(II) the carbon black (151 milligrams) and the 2,9,16,23-tetra-tert-butylphthalocyanine copper particles (97 milligrams) were mixed in the first solvent;
(III) the crystalline copper phthalocyanine-carbon black hybrid catalyst 21 was provided using ethanol as the first solvent;
(IV) the voltage was applied in such a manner that the electric potential of the cathode electrode 13 with regard to the reference electrode 19 was −1.7 volts.

The 2,9,16,23-tetra-tert-butylphthalocyanine copper particles exhibited diffraction peaks at 5.2° (lattice constant: 1.70 nanometers) and 6.0° (lattice constant: 1.48 nanometers) within a Bragg angle 2θ range of not less than 5° and not more than 10° in a powder X-ray diffraction method using a CuKα ray. See FIG. 12.

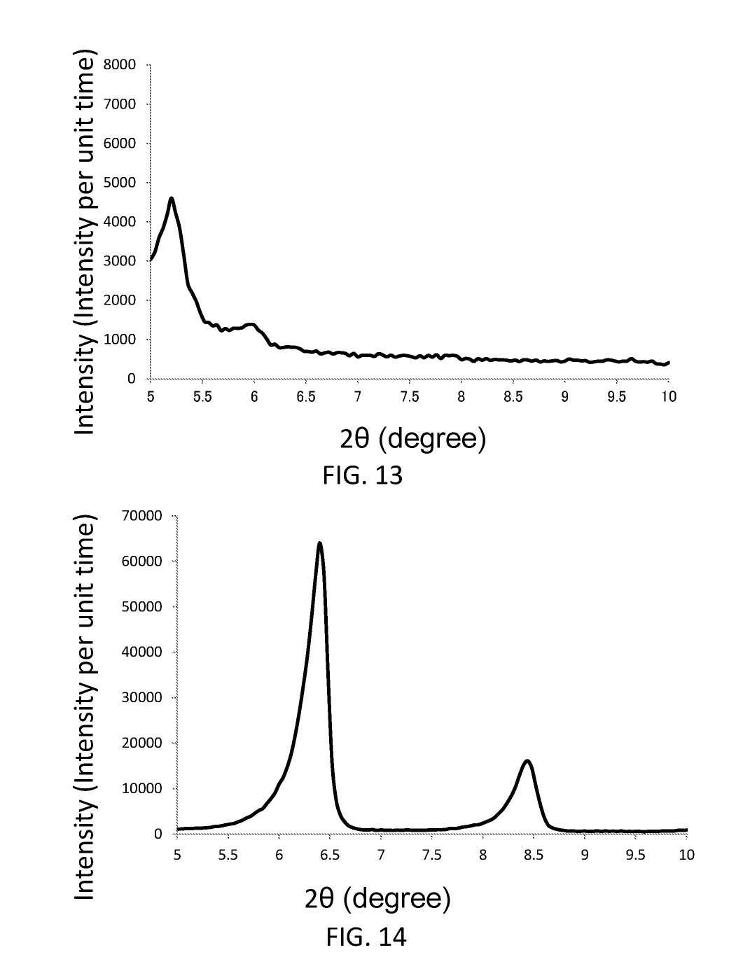

The crystallinity of the catalyst applied on the glassy carbon substrate was evaluated in the power X-ray diffraction method using the CuKα ray. As a result, as shown in FIG. 13, the diffraction peaks appeared at 5.2° (lattice constant: 1.70 nanometers, half maximum full-width: 0.40°) and 6.0° (lattice constant: 1.48 nanometers, half maximum full-width: 0.48°) within a Bragg angle 2θ range of not less than 5° and not more than 10°. Therefore, the present inventors confirmed that the copper phthalocyanine contained in the catalyst was a 2,9,16,23-tetra-tert-butylphthalocyanine copper crystal.

As a result of the experiment, the generation ratio of ethylene was 37%. See Table 1.

### Inventive Example 7

An experiment similar to the inventive example 1 was conducted, except that:

(I) 5,9,14,18,23,27,32,36-octabutoxy-2,3-naphthalocyanine copper particles (purchased from Tokyo Chemical Industry Co., Ltd.) were used as the copper phthalocyanine particles;
(II) the carbon black (68 milligrams) and the 5,9,14,18,23,27,32,36-octabutoxy-2,3-naphthalocyanine copper particles (71 milligrams) were mixed in the first solvent;
(III) the crystalline copper phthalocyanine-carbon black hybrid catalyst 21 was provided using ethanol as the first solvent;
(IV) the voltage was applied in such a manner that the electric potential of the cathode electrode 13 with regard to the reference electrode 19 was −1.7 volts.

The 5,9,14,18,23,27,32,36-octabutoxy-2,3-naphthalocyanine copper particles exhibited diffraction peaks at 6.4° (lattice constant: 1.38 nanometers) and 8.4° (lattice constant: 1.05 nanometers) within a Bragg angle 2θ range of not less than 5° and not more than 10° in a powder X-ray diffraction method using a CuKα ray. See FIG. 14.

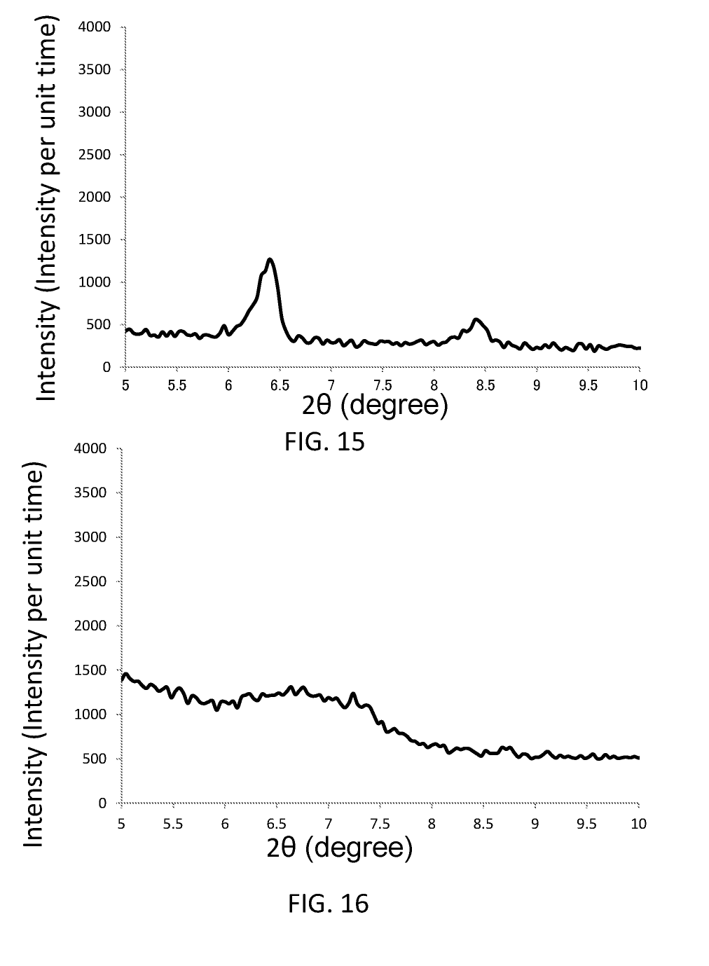

The crystallinity of the catalyst applied on the glassy carbon substrate was evaluated in the power X-ray diffraction method using the CuKα ray. As a result, as shown in FIG. 15, the diffraction peaks appeared at 6.4° (lattice constant: 1.38 nanometers, half maximum full-width: 0.24°) and 8.4° (lattice constant: 1.05 nanometers, half maximum full-width: 0.24°) within a Bragg angle 2θ range of not less than 5° and not more than 10°. Therefore, the present inventors confirmed that the copper phthalocyanine contained in the catalyst was a 5,9,14,18,23,27,32,36-octabutoxy-2,3-naphthalocyanine copper crystal.

As a result of the experiment, the generation ratio of ethylene was 30%. See Table 1.

### Comparative Example 1

An experiment similar to the inventive example 1 was conducted, except that:

(I) amorphous copper phthalocyanine was used as copper phthalocyanine contained in the catalyst; and
(II) ethyl acetate was used as both of the first solvent and the second solvent, since the crystalline copper phthalocyanine-carbon black hybrid catalyst 21 and the ink solution were prepared concurrently.

The amorphous copper phthalocyanine was prepared as below. First, the copper phthalocyanine β-type crystalline powders (125 milligrams, purchased from Tokyo Chemical Industry Co., Ltd.) were added to concentrated sulfuric acid (2 grams). Then, the mixture was stirred for one hour. Subsequently, the whole of the mixture containing the sulfuric acid and the copper phthalocyanine β-type crystalline powders was dropped to ultrapure water (12.5 milliliters). The mixture solution was stirred for thirty minutes. The mixture solution was filtrated under reduced pressure and washed. In this way, amorphous copper phthalocyanine powders were provided.

The crystallinity of the catalyst applied on the glassy carbon substrate was evaluated in the power X-ray diffraction method using the CuKα ray. As a result, as shown in FIG. 16, diffraction peaks did not appear within a Bragg angle 2θ range of not less than 5° and not more than 10°. Therefore, the present inventors confirmed that the copper phthalocyanine was amorphous.

As a result of the experiment, the generation ratio of ethylene was 17%. In other words, the ratio of the generation amount of ethylene to the whole of the products in the comparative example 1 is smaller than those of the inventive examples 1-8. See Table 1.

### Comparative Example 2

An experiment similar to the inventive example 1 was conducted, except that:

(I) amorphous 5,9,14,18,23,27,32,36-octabutoxy-2,3-naphthalocyanine copper was used as copper phthalocyanine contained in the catalyst;
(II) ethyl acetate was used as both of the first solvent and the second solvent, since the crystalline copper phthalocyanine-carbon black hybrid catalyst 21 and the ink solution were prepared concurrently;
(III) the carbon black (9.7 milligrams) and the amorphous 5,9,14,18,23,27,32,36-octabutoxy-2,3-naphthalocyanine copper particles (10 milligrams) were mixed in a solvent; and
(IV) the voltage was applied in such a manner that the electric potential of the cathode electrode 13 with regard to the reference electrode 19 was −1.7 volts.

The amorphous copper phthalocyanine was prepared as below. First, the amorphous 5,9,14,18,23,27,32,36-octabutoxy-2,3-naphthalocyanine copper powders (211 milligrams, purchased from Tokyo Chemical Industry Co., Ltd.) were added to concentrated sulfuric acid (3.4 grams). Then, the mixture was stirred for one hour. Subsequently, the whole of the mixture containing the sulfuric acid and the 5,9,14,18,23,27,32,36-octabutoxy-2,3-naphthalocyanine copper powders was dropped to ultrapure water (21.1 milliliters). The mixture solution was stirred for thirty minutes. The mixture solution was filtrated under reduced pressure and washed. In this way, the amorphous 5,9,14,18,23,27,32,36-octabutoxy-2,3-naphthalocyanine copper powders were provided.

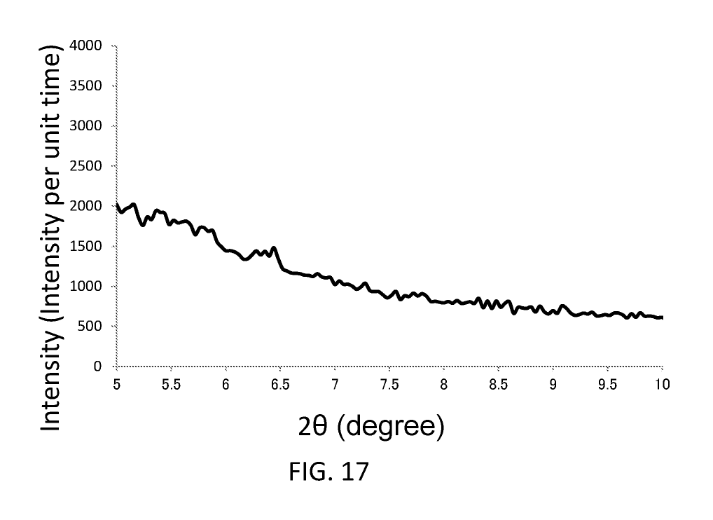

The crystallinity of the catalyst applied on the glassy carbon substrate was evaluated in the power X-ray diffraction method using the CuKα ray. As a result, as shown in FIG. 17, diffraction peaks did not appear within a Bragg angle 2θ range of not less than 5° and not more than 10°. Therefore, the present inventors confirmed that the 5,9,14,18,23,27,32,36-octabutoxy-2,3-naphthalocyanine copper was amorphous.

As a result of the experiment, the generation ratio of ethylene was 9.8%. In other words, the ratio of the generation amount of ethylene to the whole of the products in the comparative example 2 is smaller than those of the inventive examples 1-8. See Table 1.

### Comparative Example 3

An experiment similar to the inventive example 1 was conducted, except that:

(I) only the carbon black was used without copper phthalocyanine;
(II) the dispersion in the first solvent was not conducted;
(III) acetone was used as the second solvent to prepare the ink solution; and
(IV) the voltage was applied in such a manner that the electric potential of the cathode electrode 13 with regard to the reference electrode 19 was −1.7 volts.

As a result of the experiment, the generation ratio of ethylene was not more than 2.0%. In other words, ethylene was seldom generated.

The following Table 1 shows the generation ratio of each of the products in the inventive examples 1-7 and the comparative examples 1-3. The following Table 2 shows the generation amount thereof.

TABLE 1
Generation ratio (%)

| | H₂ | CO | CH₄ | C₂H₄ | HCOOH |
|---|---|---|---|---|---|
| Inventive example 1 | 26 | 9.7 | 1.2 | 41 | 22 |
| Inventive example 2 | 30 | 11 | 1.3 | 31 | 26 |
| Inventive example 3 | 31 | 1.8 | 13 | 42 | 12 |
| Inventive example 4 | 17 | 4.8 | 22 | 43 | 13 |
| Inventive example 5 | 22 | 2.9 | 25 | 38 | 12 |
| Inventive example 6 | 29 | 1.9 | 19 | 37 | 13 |
| Inventive example 7 | 24 | 7.8 | 16 | 30 | 22 |
| Comparative example 1 | 42 | 20 | 1.5 | 17 | 19 |
| Comparative example 2 | 34 | 9.3 | 5.9 | 9.8 | 41 |
| Comparative example 3 | 17 | 30 | 1.9 | 1.8 | 50 |

TABLE 2
Generation amount (micromol)

| | H₂ | CO | CH₄ | C₂H₄ | HCOOH |
|---|---|---|---|---|---|
| Inventive example 1 | 123 | 45 | 1 | 32 | 102 |
| Inventive example 2 | 131 | 49 | 1 | 22 | 114 |
| Inventive example 3 | 343 | 19 | 37 | 77 | 131 |
| Inventive example 4 | 75 | 21 | 24 | 32 | 57 |
| Inventive example 5 | 147 | 19 | 41 | 42 | 77 |
| Inventive example 6 | 276 | 18 | 45 | 57 | 122 |
| Inventive example 7 | 87 | 28 | 14 | 18 | 77 |
| Comparative example 1 | 99 | 48 | 1 | 7 | 45 |
| Comparative example 2 | 75 | 21 | 3 | 4 | 91 |
| Comparative example 3 | 38 | 68 | 1 | 1 | 115 |

As shown in Table 1 and Table 2, ethylene was generated selectively, since the crystalline copper phthalocyanine-carbon black hybrid catalyst 21 was used as the cathode electrode. In other words, this means that copper phthalocyanine having crystallinity included in the carbon black and capable of reducing carbon dioxide electrochemically contributes to the selectivity in the generation amount of ethylene.

### INDUSTRIAL APPLICABILITY

The present disclosure provides a method for reducing carbon dioxide electrochemically to generate ethylene selectively. The present disclosure also provides a method for generating ethylene selectively. The present disclosure further provides an electrolysis device, a carbon dioxide reduction electrode, and a carbon dioxide reduction catalyst used therefor.

### REFERENTIAL SIGN LIST

1 Pipe
11 First electrolyte solution
12 Cathode container
13 Cathode electrode
14 Second electrolyte solution
15 Anode container
16 Solid electrolyte membrane
17 Anode electrode
18 External power source
19 Reference electrode
21 Crystalline copper phthalocyanine-carbon black hybrid catalyst
22 Electrically conductive layer
23 Substrate
41 Layer
42 Layer
43 Glassy carbon substrate
100 Electrolysis device

1. A method for reducing carbon dioxide electrochemically to generate ethylene selectively, the method comprising:
(a) preparing an electrolysis device comprising:
a cathode container;
an anode container;
a cathode electrode;
an anode electrode; and
a solid electrolysis membrane;
wherein
a first electrolysis solution is stored in the cathode container;
the first electrolysis solution contains the carbon dioxide;
a second electrolysis solution is stored in the anode container;
the cathode electrode is in contact with the first electrolysis solution;
the cathode electrode comprises a carbon dioxide reduction catalyst;
the carbon dioxide reduction catalyst comprises a crystalline copper phthalocyanine;
the anode electrode is in contact with the second electrolysis solution; and
the cathode container and the anode container are separated from each other with the solid electrolysis membrane; and
(b) applying a voltage to the cathode electrode and the anode electrode in such a manner that an electric potential of the cathode electrode is negative with regard to an electric potential of the anode electrode to generate ethylene selectively due to electrochemical reduction of the carbon dioxide on the cathode electrode.

2. The method according to claim 1, wherein
at least a part of the crystalline copper phthalocyanine is α-type crystalline.

3. The method according to claim 1, wherein
at least a part of the crystalline copper phthalocyanine is β-type crystalline.

4. The method according to claim 1, wherein
at least a part of the crystalline copper phthalocyanine is a 1,2,3,4,8,9,10,11,15,16,17,18,22,23,24,25-hexadecafluorophthalocyanine copper crystal.

5. The method according to claim 1, wherein
at least a part of the crystalline copper phthalocyanine is a 2,3,9,10,16,17,23,24-octafluorophthalocyanine copper crystal.

6. The method according to claim 1, wherein
at least a part of the crystalline copper phthalocyanine is a 2,9,16,23-tetra-tert-butyl phthalocyanine copper crystal.

7. The method according to claim 1, wherein
at least a part of the crystalline copper phthalocyanine is a 5,9,14,18,23,27,32,36-octabutoxy-2,3-naphthalocyanine copper crystal.

8. An electrolysis device for generating ethylene selectively due to reduction of carbon dioxide electrochemically; the electrolysis device comprising:
a cathode container;
an anode container;
a cathode electrode;
an anode electrode; and
a solid electrolysis membrane;
wherein
a first electrolysis solution is stored in the cathode container;
the first electrolysis solution contains the carbon dioxide;
a second electrolysis solution is stored in the anode container;
the cathode electrode is in contact with the first electrolysis solution;
the cathode electrode comprises a carbon dioxide reduction catalyst;
the carbon dioxide reduction catalyst comprises a crystalline copper phthalocyanine;
the anode electrode is in contact with the second electrolysis solution; and
the cathode container and the anode container are separated from each other with the solid electrolysis membrane.

9. The electrolysis device according to claim 8, wherein
at least a part of the crystalline copper phthalocyanine is α-type crystalline.

10. The electrolysis device according to claim 8, wherein
at least a part of the crystalline copper phthalocyanine is β-type crystalline.

11. The electrolysis device according to claim 8, further comprising
a reference electrode disposed in the cathode container, wherein
the reference electrode is in contact with the first electrolysis solution; and
the reference electrode has a region of silver/silver chloride.

12. The electrolysis device according to claim 8, wherein
the anode electrode is formed of a material selected from the group consisting of carbon, platinum, gold, silver, copper, titanium, iridium oxide, and an alloy thereof.

13. The electrolysis device according to claim 8, wherein
at least a part of the crystalline copper phthalocyanine is a 1,2,3,4,8,9,10,11,15,16,17,18,22,23,24,25-hexadecafluorophthalocyanine copper crystal.

14. The electrolysis device according to claim 8, wherein
at least a part of the crystalline copper phthalocyanine is a 2,3,9,10,16,17,23,24-octafluorophthalocyanine copper crystal.

15. The electrolysis device according to claim 8, wherein
at least a part of the crystalline copper phthalocyanine is a 2,9,16,23-tetra-tert-butyl phthalocyanine copper crystal.

16. The electrolysis device according to claim 8, wherein
at least a part of the crystalline copper phthalocyanine is a 5,9,14,18,23,27,32,36-octabutoxy-2,3-naphthalocyanine copper crystal.

17. A carbon dioxide reduction electrode capable of converting carbon dioxide into ethylene selectively by application of a voltage, comprising:
crystalline copper phthalocyanine.

18. The carbon dioxide reduction electrode according to claim 17, wherein
at least a part of the crystalline copper phthalocyanine is α-type crystalline.

19. The carbon dioxide reduction electrode according to claim 17, wherein
at least a part of the crystalline copper phthalocyanine is β-type crystalline.

20. The carbon dioxide reduction electrode according to claim 17, wherein
at least a part of the crystalline copper phthalocyanine is a 1,2,3,4,8,9,10,11,15,16,17,18,22,23,24,25-hexadecafluorophthalocyanine copper crystal.

21. The carbon dioxide reduction electrode according to claim 17, wherein
at least a part of the crystalline copper phthalocyanine is a 2,3,9,10,16,17,23,24-octafluorophthalocyanine copper crystal.

22. The carbon dioxide reduction electrode according to claim 17, wherein
at least a part of the crystalline copper phthalocyanine is a 2,9,16,23-tetra-tert-butyl phthalocyanine copper crystal.

23. The carbon dioxide reduction electrode according to claim 17, wherein
at least a part of the crystalline copper phthalocyanine is a 5,9,14,18,23,27,32,36-octabutoxy-2,3-naphthalocyanine copper crystal.

24. A carbon dioxide reduction catalyst comprising:
crystalline copper phthalocyanine.

25. The carbon dioxide reduction catalyst according to claim 24, further comprising
carbon black, wherein
the crystalline copper phthalocyanine is mixed with the carbon black.

26. The carbon dioxide reduction catalyst according to claim 24, wherein
at least a part of the crystalline copper phthalocyanine is α-type crystalline.

27. The carbon dioxide reduction catalyst according to claim 24, wherein
at least a part of the crystalline copper phthalocyanine is β-type crystalline.

28. The carbon dioxide reduction catalyst according to claim 24, wherein
at least a part of the crystalline copper phthalocyanine is a 1,2,3,4,8,9,10,11,15,16,17,18,22,23,24,25-hexadecafluorophthalocyanine copper crystal.

29. The carbon dioxide reduction catalyst according to claim 24, wherein
at least a part of the crystalline copper phthalocyanine is a 2,3,9,10,16,17,23,24-octafluorophthalocyanine copper crystal.

30. The carbon dioxide reduction catalyst according to claim 24, wherein
at least a part of the crystalline copper phthalocyanine is a 2,9,16,23-tetra-tert-butyl phthalocyanine copper crystal.

31. The carbon dioxide reduction electrode according to claim 24, wherein
at least a part of the crystalline copper phthalocyanine is a 5,9,14,18,23,27,32,36-octabutoxy-2,3-naphthalocyanine copper crystal.
